(19) United States
(12) Patent Application Publication
(10) Pub. No.: US 2024/0148302 A1
(43) Pub. Date: May 9, 2024

(54) ELECTRODE AND METHOD FOR PRODUCING THE SAME

(71) Applicants: Murata Manufacturing Co., Ltd., Nagaokakyo-shi (JP); The Trustees of the University of Pennsylvania, Philadelphia, PA (US)

(72) Inventors: Brendan Boyce MURPHY, Nagaokakyo-shi (JP); Flavia Vitale, Wilmington, DE (US); Mika Fujiwaki, Nagaokakyo-shi (JP); Takeshi Torita, Nagaokakyo-shi (JP); Kosuke Sugiura, Nagaokakyo-shi (JP); Toshiko Shimazaki, Nagaokakyo-shi (JP)

(21) Appl. No.: 18/413,810

(22) Filed: Jan. 16, 2024

Related U.S. Application Data
(63) Continuation of application No. PCT/JP22/27742, filed on Jul. 14, 2022.
(60) Provisional application No. 63/223,306, filed on Jul. 19, 2021.

(51) Int. Cl. A61B 5/268; A61B 5/28; A61B 5/296

(52) U.S. Cl. CPC A61B 5/268; A61B 5/28; A61B 5/296

(57) ABSTRACT

An electrode comprising: a π electron conjugated compound film which includes a π electron conjugated compound having at least one of an aromatic ring, a heteroaromatic ring, a carbon-carbon double bond, a carbon-carbon triple bond, and a carbon-phosphorus double bond; and a conductive film bonded to the r electron conjugated compound film, the conductive film including particles of a layered material of one or plural layers having a layer body represented by: MₘXₙ, wherein M is at least one metal of Group 3 through 7, X is a carbon atom, a nitrogen atom, or a combination thereof, n is 1 to 4, and m is more than n but not more than 5, and a modifier or terminal T existing on a surface of the layer body, wherein T is at least one of a hydroxyl group, a fluorine atom, a chlorine atom, an oxygen atom, and a hydrogen atom.

### CROSS REFERENCE TO RELATED APPLICATIONS

The present application is a continuation of International application No. PCT/JP2022/027742, filed Jul. 14, 2022, which claims priority to U.S. Provisional Patent Application No. 63/223,306, filed Jul. 19, 2021, the entire contents of each of which are incorporated herein by reference.

### TECHNICAL FIELD

The present disclosure relates to an electrode and a method for producing the same.

### BACKGROUND ART

In recent years, MXene has been attracting attention as a new material having conductivity. MXene is a type of so-called two-dimensional material, and as will be described later, is a layered material in the form of one or plural layers. In general, MXene is in the form of particles (which can include powders, flakes, nanosheets, and the like) of such a layered material.

Currently, various studies are being conducted toward the application of MXene to various electric devices. For example, WO 2019/055784 A (“Patent Literature 1”) discloses an electrode having a contact material containing MXene and exhibiting impedance lower than that of Au as an electric device.

### SUMMARY OF THE INVENTION

However, since the conductivity of MXene may be deteriorated over time (for example, in about several days to one month), it has been required to maintain excellent electrode characteristics such as high conductivity for a long period of time. The present disclosure has been made in view of the above circumstances, and an object of the present disclosure is to provide an electrode capable of maintaining excellent electrode characteristics for a long period of time, and a method for producing the same.

According to one aspect of the present invention, there is provided an electrode comprising:
a π-electron conjugated compound film which comprises a π electron conjugated compound having at least one selected from the group consisting of an aromatic ring, a heteroaromatic ring, a carbon-carbon double bond, a carbon-carbon triple bond, and a carbon-phosphorus double bond; and
a conductive film bonded to the n electron conjugated compound film, wherein the conductive film comprises particles of a layered material comprising one or plural layers, the one or plural layers comprising a layer body represented by:

MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is at least one selected from the group consisting of a hydroxyl group, a fluorine atom, a chlorine atom, an oxygen atom, and a hydrogen atom.

According to another aspect of the present invention, there is provided a method for producing an electrode, the method comprising:
(a) preparing a precursor of a conductive film, the precursor represented by:

MₘAXₙ
wherein M is at least one metal of Group 3, 4, 5, 6, or 7, X is a carbon atom, a nitrogen atom, or a combination thereof, A is at least one element of Group 12, 13, 14, 15, or 16, n is not less than 1 and not more than 4, and m is more than n but not more than 5;
(b) removing at least a part of the A atoms from the precursor using an etching liquid to obtain a first intermediate;
(c) washing the first intermediate with water to obtain a second intermediate;
(d) performing intercalation by mixing the second intermediate with a compound for inter-layer insertion of the second intermediate to obtain a third intermediate;
(e) stirring the third intermediate in a liquid to obtain a fourth intermediate;
(f) washing the fourth intermediate with water to obtain particles of a layered material;
(g) forming a conductive film comprising the particles of the layered material on a substrate; and
(h) forming, on the conductive film, a r-electron conjugated compound film which comprises a π-electron conjugated compound having at least one selected from the group consisting of an aromatic ring, a heteroaromatic ring, a carbon-carbon double bond, a carbon-carbon triple bond, and a carbon-phosphorus double bond.

According to the present invention, an electrode includes a laminate of a conductive film containing particles of a predetermined layered material (also referred to as “MXene” in the present specification) including one or more layers and a π-electron conjugated compound film, thereby maintaining stable electrode characteristics for a long period of time with MXene.

### BRIEF DESCRIPTION OF THE DRAWINGS

FIGS. 1(a) and 1(b) are schematic cross-sectional views illustrating MXene which is a layered material usable for a conductive film of an electrode in the present embodiment, in which FIG. 1(a) illustrates single-layered MXene, and FIG. 1(b) illustrates multi-layered (exemplarily two-layered) MXene.

FIG. 2 is a schematic cross-sectional view for explaining an action and effect of one embodiment of the electrode of the present embodiment.

### DETAILED DESCRIPTION OF THE PREFERRED EMBODIMENTS

### Embodiment 1: Electrode

Hereinafter, an electrode in one embodiment of the present invention will be described in detail, but the present invention is not limited to such an embodiment.

According to the present embodiment, an electrode includes:
a π-electron conjugated compound film which comprises a π electron conjugated compound having at least one selected from the group consisting of an aromatic ring, a heteroaromatic ring, a carbon-carbon double bond, a carbon-carbon triple bond, and a carbon-phosphorus double bond; and
a conductive film bonded to the n electron conjugated compound film, wherein the conductive film contains particles of a layered material including one or plural layers, wherein the one or plural layers including a layer body represented by:

MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is at least one selected from the group consisting of a hydroxyl group, a fluorine atom, a chlorine atom, an oxygen atom, and a hydrogen atom.

The electrode can maintain stable electrode characteristics for a long period of time by having the conductive film. For example, low impedance can be secured as electrode characteristics, for example. More specifically, for example, under a biological environment, it is possible to suppress an increase in initial impedance of the electrode and to suppress an increase in impedance over time.

The layered material can be understood as a layered compound and is also denoted “MₘXₙTₛ”, in which s is an optional number, and in the related art, x or z may be used instead of s. Typically, n may be, but is not limited to, 1, 2, 3, or 4.

In the above formula of MXene, M is preferably at least one selected from the group consisting of Ti, Zr, Hf, V, Nb, Ta, Cr, Mo, and Mn, and more preferably at least one selected from the group consisting of Ti, V, Cr, and Mo.

MXene is known that the above formula: MₘXₙ is expressed as follows: Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti,V)₂C, (Ti,Nb)₂C, W₂C, W_{1.3}C, Mo₂N, Nb_{1.3}C, Mo_{1.3}Y_{0.6}C (in the above formula, “1.3” and “0.6” mean about 1.3 (= 4/3) and about 0.6 (=⅔), respectively); Ti₃C₂, Ti₃N₂, Ti₃ (CN), Zr₃C₂, (Ti,V)₃C₂, (Ti₂Nb) C₂, (Ti₂Ta) C₂, (Ti₂Mn) C₂, Hf₃C₂, (Hf₂V) C₂, (Hf₂Mn) C₂, (V₂Ti) C₂, (Cr₂Ti)C₂, (Cr₂V)C₂, (Cr₂Nb)C₂, (Cr₂Ta)C₂, (Mo₂Sc)C₂, (Mo₂Ti)C₂, (Mo₂Zr) C₂, (Mo₂Hf) C₂, (Mo₂V) C₂, (Mo₂Nb) C₂, (Mo₂Ta) C₂, (W₂Ti) C₂, (W₂Zr) C₂, (W₂Hf) C₂; Ti₄N₃, V₄C₃, Nb₄C₃, Ta₄C₃, (Ti,Nb)₄C₃, (Nb, Zr)₄C₃, (Ti₂Nb₂) C₃, (Ti₂Ta₂) C₃, (V₂Ti₂) C₃, (V₂Nb₂) C₃, (V₂Ta₂) C₃, (Nb₂Ta₂) C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, (W₂Hf₂)C₃, (Mo_{2.7}V_{1.3})C₃ (in the above formula, “_{2.7}” and “_{1.3}” mean about 2.7 (= 8/3) and about 1.3 (= 4/3), respectively.)

Typically, in the above formula, M may be titanium or vanadium and X may be a carbon atom or a nitrogen atom. For example, the MAX phase that is a precursor of MXene is Ti₃AlC₂, and MXene is Ti₃C₂Tₛ(in other words, M is Ti, X is C, n is 2, and m is 3).

In the present embodiment, MXene may contain a relatively small amount of remaining A atoms, for example, 10% by mass or less with respect to the original A atoms. The residual amount of A atoms may be preferably 8% by mass or less, and more preferably 6% by mass or less. However, even if the residual amount of A atoms exceeds 10% by mass, there may be no problem depending on the application and use conditions of the electrode.

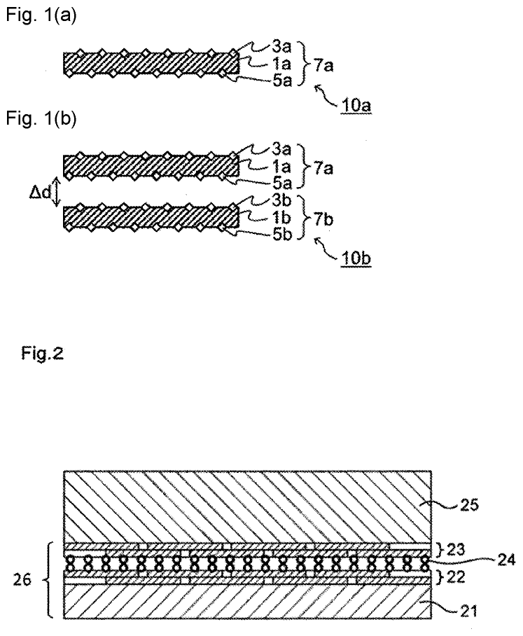

Hereinafter, particles of a layered material (also referred to as “MXene particles”) constituting the conductive film in the electrode according to the present embodiment will be described with reference to FIGS. 1(a) and 1(b).

The electrode of the present embodiment is an aggregate containing one layer of MXene 10a (single layer MXene) schematically illustrated in FIG. 1(a). More specifically, MXene 10a is an MXene layer 7a having layer body (MₘXₙ layer) 1a represented by MₘXₙ, and modifier or terminals T 3a and 5a existing on the surface (more specifically, at least one of two surfaces facing each other in each layer) of the layer body 1a. Therefore, the MXene layer 7a is also represented as “MₘXₙTₛ”, and s is an optional number.

The conductive film according to the present embodiment may include one or plural layers. Examples of the MXene (multilayer MXene) of the plural layers include, but are not limited to, two layers of MXene 10b as schematically illustrated in FIG. 1 (b). 1b, 3b, 5b, and 7b in FIG. 1 (b) are the same as 1a, 3a, 5a, and 7a in FIG. 1(a) described above. Two adjacent MXene layers (for example, 7a and 7b) of the multilayer MXene do not necessarily have to be completely separated from each other, and may be partially in contact with each other. The MXene 10a may be one in which the multilayer MXene 10b are individually separated and exist in one layer. The MXene 10 may be a mixture of the single layer MXene 10a and the multilayer MXene 10b in which the unseparated multilayer MXene 10b remains. Even when the multilayer MXene is included, the multilayer MXene is preferably MXene having a few-layer obtained through the delamination treatment. The term “the number of layers is small” means that, for example, the number of stacked layers of MXene is not more than ten. Hereinafter, the “multilayer MXene having a few-layer” may be referred to as a “few-layer MXene” in some cases. The thickness of the few-layer MXene in the lamination direction is preferably not more than 10 nm. In addition, the single layer MXene and the few-layer MXene may be collectively referred to as “single layer/few-layer MXene” in some cases.

The conductive film according to the present embodiment preferably contains a large amount of single layer/few-layer MXene. By containing a large amount of the single layer/few-layer MXene, the specific surface area of MXene can be made larger than that of the multilayer MXene, and as a result, as shown in examples described later, deterioration of electrode characteristics over time can be further suppressed. For example, in the conductive film according to the present embodiment, the single layer/few-layer MXene, in which the number of laminated layers of MXene is not more than 10 layers and the thickness is preferably not more than 10 nm, accounts for preferably not less than 80% by volume, more preferably not less than 90% by volume, and still more preferably not less than 95% by volume of the total MXene. In addition, the volume of the single layer MXene is more preferably larger than the volume of the few-layer MXene. Since the true density of these MXenes does not greatly vary depending on the existence form, it can be said that it is more preferable that the mass of the single layer MXene is larger than the mass of the few-layer MXene. When these relationships are satisfied, the specific surface area can be further increased, and the deterioration of the electrode characteristics over time can be further suppressed. Most preferably, the conductive film according to the present embodiment is formed only of the single layer MXene.

Although the present embodiment is not limited, the thickness of each layer of MXene (which corresponds to the MXene layers 7a and 7b) is, for example, not less than 0.8 nm and not more than 5 nm, particularly not less than 0.8 nm and not more than 3 nm (which may mainly vary depending on the number of M atom layers included in each layer). For the individual stacked films of the multilayer MXene that can be included, the interlayer distance (alternatively, a void dimension is indicated by Ad in FIG. 1(b)) is, for example, not less than 0.8 nm and not more than 10 nm, particularly not less than 0.8 nm and not more than 5 nm, and more particularly about 1 nm, and the total number of layers can be not less than 2 and not more than 20,000.

In the electrode of the present embodiment, the conductive film and the π-electron conjugated compound film are stacked to form a stacked film. FIG. 2 is a schematic cross-sectional view in a case where a substrate 21 is included and a biological tissue 25 is used as a subject as an embodiment of the electrode of the present embodiment. Examples of the measurement target in the biological tissue 25 include blood vessels, muscles, brains, and other organs under the skin in addition to the skin such as a human body. The measurement may be performed by bringing an electrode into direct contact with the measurement target, or for example, the measurement target under the skin may be measured indirectly by bringing an electrode into contact with the skin. In particular, in FIG. 2, a bond 24 between the r electrons of the MXene particles constituting the conductive film 22 and the n electrons of the π-electron conjugated compound film 23 is exaggeratedly shown for the description of the action and effect, and originally, a gap formed by the bond 24 in FIG. 2 is not formed between the conductive film 22 and the π-electron conjugated compound film 23, and the conductive film 22 and the r-electron conjugated compound film 23 are substantially in contact with each other. As illustrated in FIG. 2, by providing and covering the r-electron conjugated compound film 23 so as to be in contact with the conductive film 22, the π-electron conjugated compound film 23 can be bonded between the conductive films 22 by n electrons to secure a conductive path, and oxidation of the conductive film 22 is prevented. As a result, excellent electrode characteristics such as maintenance of high conductivity, suppression of an increase in initial impedance, and suppression of an increase in impedance over time can be realized. The biological tissue 25, for example, the skin of the human body and the electrode 26 are brought into direct contact with each other as illustrated in FIG. 2, and a gel or a film (not shown) capable of permeating ions may be present between the biological tissue 25 and the electrode 26.

The π-electron conjugated compound film contains a n-electron conjugated compound having one or more selected from the group consisting of an aromatic ring, a heteroaromatic ring, a carbon-carbon double bond, a carbon-carbon triple bond, and a carbon-phosphorus double bond. The compound has a molecular structure in which n electrons are delocalized, and is bonded to n electrons of MXene particles constituting the conductive film to exhibit the above-described action and effect. The heteroaromatic ring has a heteroatom such as nitrogen, oxygen, sulfur, phosphorus, chlorine, iodine, or bromine as a constituent element of the ring. The π-electron conjugated compound film may have a microscopically continuous form of the compound, or may be an aggregate of the compounds having a plate shape, a granular shape, a powder shape, a columnar shape, and a cylindrical shape.

The π-electron conjugated compound is preferably one or more compounds selected from the group consisting of graphene, graphene oxide, reduced graphene oxide, a carbon nanotube, polyacetylene, polyparaphenylene, polyparaphenylene vinylene, polypyrrole, polythiophene, polyethylene dioxythiophene, polyaniline, and polythienylene vinylene. More preferably, the π-electron conjugated compound is one or more compounds selected from the group consisting of graphene, graphene oxide, reduced graphene oxide, and a carbon nanotube.

The thickness of the π-electron conjugated compound film can be, for example, in a range of not less than 0.03 μm and not more than 30 μm.

In order to enhance the effect by stacking the conductive film and the π-electron conjugated compound film, the face of the conductive film on the π-electron conjugated compound film side is preferably covered with the n-electron conjugated compound film in an amount of 80% by area or more, more preferably 90% by area or more, and most preferably 100% by area or more. The side surface of the conductive film may be covered with a π-electron conjugated compound film. The oxidation of the conductive film generally easily proceeds from an edge of the conductive film (a portion near an outer shape of the conductive film). Therefore, it is preferable that the π-electron conjugated compound film also covers the edge and the side surface of the conductive film.

In addition, from the viewpoint of reliably securing the electrode characteristics inherent in the conductive film and sufficiently suppressing an increase in initial impedance and an increase in impedance over time, for example, as shown in examples described later, a ratio of the amount of the n-electron conjugated compound film to the total amount of the conductive film and the π-electron conjugated compound film is preferably 80% by mass or less. The ratio is more preferably 75% by mass or less, and still more preferably 70% by mass or less. On the other hand, in order to sufficiently exhibit the above effect by formation of the π-electron conjugated compound film, the ratio is preferably 5% by mass or more, more preferably 15% by mass or more, and still more preferably 30% by mass or more.

The number-average value of a ferret diameter of the particles of the layered material contained in the conductive film according to the present embodiment is preferably 3 μm or more. As described above, the shape of the particles of the layered material has a plane, and as the plane region is larger, the number of end faces (edges) of the particles of the layered material, which are likely to form oxidation sites, is relatively reduced, and it can be expected that the in-plane orientation of the entire coating film is improved. As a result, the contact area with the π-electron conjugated compound film is increased, a large number of n bonds between the conductive film and the π-electron conjugated compound film are easily formed, the conductivity is increased, and for example, the deterioration of the electrode characteristics over time such as an increase in the impedance over time can be further suppressed. In the present specification, the size of the planar portion of the particles of the layered material is defined by the number-average value of a ferret diameter. The number-average value of a ferret diameter is more preferably 4 μm or more. The number-average value of a ferret diameter is preferably as large as possible from the viewpoint of maintaining excellent electrode characteristics over a long period of time, but the upper limit of the number-average value of a ferret diameter is about 50 μm in consideration of production efficiency and the like. Examples of the method for measuring the ferret diameter include a method for measuring the ferret diameter at a stage of producing the MXene particles, that is, using the MXene slurry, and a method for measuring the ferret diameter of the MXene particles constituting the conductive film of the electrode by removing the r-electron conjugated compound film from the stacked film with the r-electron conjugated compound film. The number-average value of a ferret diameter can be determined using a scanning electron microscope (SEM) or an atomic force microscope (AFM) as shown in examples described later.

The electrode of the present embodiment is not limited to a specific form as long as at least the conductive film and the π-electron conjugated compound film are stacked. Examples of the electrode include an electrode in a solid state and an electrode in a flexible and soft state. The thicknesses of the conductive film and the π-electron conjugated compound film can be measured by, for example, measurement with a micrometer, cross-sectional observation by a method such as a scanning electron microscope (SEM), a microscope, or a laser microscope.

One of the characteristics of the electrode of the present embodiment is impedance. The initial impedance is preferably as small as possible, and is preferably smaller within a range of 80 kΩcm² or less at 10 Hz and 10 kΩcm² or less at 1 kHz, for example, according to measurement conditions shown in examples described later.

In the electrode of the present embodiment, the conductive film is brought into direct contact with the r-electron conjugated compound film. In the electrode of the present embodiment, the face of the π-electron conjugated compound film on the side opposite to the conductive film side may be exposed to the outside air so as to be brought into direct contact with an object to be measured, or as another laminate, a gel capable of permeating ions, a film including a porous membrane, or the like may be formed. The porous membrane may be a membrane having a large number of fine pores and capable of selectively transmitting ions and molecules having a size smaller than a pore diameter. The materials for these other laminates are not particularly limited and may be formed of organic materials, inorganic materials, or mixtures thereof. Polymers such as hydrophilic polymers as the organic materials, and ceramics as the inorganic materials, or a combination thereof can be exemplified. The film thickness of the other laminates may be, for example, not less than 0.1 μm and not more than 300 μm. The porous membrane may have, for example, an average pore diameter of not less than 1 nm and not more than 1 μm. The porous membrane may be, for example, an aggregated particulate porous membrane, a network porous membrane, a fibrous porous membrane, a porous membrane having a plurality of isolated and/or communicating pipe holes, a porous membrane having a honeycomb structure, or the like, depending on the pore shape.

When the electrode of the present embodiment includes a substrate, the conductive film and the substrate may be brought into direct contact with each other. The material of the substrate is not particularly limited. The substrate may be formed of a conductive material. Examples of the conductive material include at least one material of metal materials such as gold, silver, copper, platinum, nickel, titanium, tin, iron, zinc, magnesium, aluminum, tungsten, and molybdenum, and a conductive polymer. The substrate may include a conductive film, such as a metal film, different from the conductive film according to the present embodiment on a contact surface with the conductive film according to the present embodiment. Alternatively, the substrate may be formed of an organic material. Examples of the organic material include flexible organic materials, such as a thermoplastic polyurethane elastomer (TPU), a PET film, and a polyimide film.

(Application of Electrode)

The electrode of the present embodiment can be used for any appropriate application. For example, it may be used in applications where maintaining high conductivity (to reduce a decrease in initial conductivity and prevent oxidation) is required, such as electrodes or electromagnetic shielding (EMI shielding) in any suitable electric device.

The electrode is not particularly limited, and may be, for example, a capacitor electrode, a battery electrode, a biosignal sensing electrode, a sensor electrode, an antenna electrode, or the like. By using the conductive film of the present embodiment, it is possible to obtain a large-capacity capacitor and battery, a low-impedance biosignal sensing electrode, a highly sensitive sensor, and an antenna even with a smaller volume (device occupied volume).

The capacitor may be an electrochemical capacitor. The electrochemical capacitor is a capacitor using capacitance developed due to a physicochemical reaction between an electrode (electrode active material) and ions (electrolyte ions) in an electrolytic solution, and can be used as a device (power storage device) that stores electric energy. The battery may be a repeatedly chargeable and dischargeable chemical battery. The battery may be, for example, but not limited to, a lithium ion battery, a magnesium ion battery, a lithium sulfur battery, a sodium ion battery, or the like.

The biosignal sensing electrode is an electrode for acquiring a biological signal. The biosignal sensing electrode may be, for example, but not limited to, an electrode for measuring electroencephalogram (EEG), electrocardiogram (ECG), electromyogram (EMG), electrical impedance tomography (EIT).

The sensor electrode is an electrode for detecting a target substance, state, abnormality, or the like. The sensor may be, for example, but not limited to, a gas sensor, a biosensor (a chemical sensor utilizing a molecular recognition mechanism of biological origin), or the like.

The antenna electrode is an electrode for emitting an electromagnetic wave into a space and/or receiving an electromagnetic wave in the space.

The electrode of the present embodiment is preferably used as a biosignal sensing electrode in which the n-electron conjugated compound film is provided on a contact surface with a subject.

### Embodiment 2: Method for Producing Electrode

Hereinafter, a method for producing an electrode in the embodiment of the present invention will be described in detail, but the present invention is not limited to such an embodiment.

According to the present embodiment, a method for producing an electrode includes
(a) preparing a precursor of a conductive film represented by:

MₘAXₙ
wherein M is at least one metal of Group 3, 4, 5, 6, or 7, X is a carbon atom, a nitrogen atom, or a combination thereof, A is at least one element of Group 12, 13, 14, 15, or 16, n is not less than 1 and not more than 4, and m is more than n but not more than 5;
(b) removing at least a part of the A atoms from the precursor using an etching liquid to obtain a first intermediate;
(c) washing the first intermediate with water to obtain a second intermediate;
(d) performing an intercalation by mixing the second intermediate with a compound for inter-layer insertion of the second intermediate to obtain a third intermediate;
(e) stirring the third intermediate in a liquid to obtain a fourth intermediate;
(f) washing the fourth intermediate with water in order to obtain particles of a layered material;
(g) forming a conductive film containing the particles of the layered material on a substrate; and
(h) forming a π-electron conjugated compound film containing a π-electron conjugated compound having one or more selected from the group consisting of an aromatic ring, a heteroaromatic ring, a carbon-carbon double bond, a carbon-carbon triple bond, and a carbon-phosphorus double bond on the conductive film.

By this producing method, it is possible to produce an electrode capable of maintaining excellent electrode characteristics for a long period of time.

Hereinafter, each step of the production method will be described in detail.

### Step (a)

First, a predetermined precursor is prepared. A predetermined precursor that can be used in the present embodiment is a MAX phase that is a precursor of MXene, and is represented by a formula below:

MₘAXₙ
wherein M is at least one metal of Group 3, 4, 5, 6, or 7, X is a carbon atom, a nitrogen atom, or a combination thereof, A is at least one element of Group 12, 13, 14, 15, or 16, n is not less than 1 and not more than 4, and m is more than n but not more than 5.

The above M, X, n, and m are as described in MXene. A is at least one element of Group 12, 13, 14, 15, or 16, is usually a Group A element, typically Group IIIA and Group IVA, more specifically, may include at least one selected from the group consisting of Al, Ga, In, Tl, Si, Ge, Sn, Pb, P, As, S, and Cd, and is preferably Al.

The MAX phase has a crystal structure in which a layer constituted by A atoms is located between two layers represented by MₘXₙ (each X may have a crystal lattice located in an octahedral array of M). Typically, in the case of m=n+1, the MAX phase has a repeating unit in which one layer of X atoms is disposed between the layers of M atoms of n+1 layers (these layers are also collectively referred to as “MₘXₙ layer”), and a layer of A atoms (“A atom layer”) is disposed as a next layer of the (n+1) th layer of M atoms; however, the present invention is not limited thereto.

The MAX phase can be produced by a known method. For example, a TiC powder, a Ti powder, and an Al powder are mixed in a ball mill, and the obtained mixed powder is fired under an Ar atmosphere to obtain a fired body (block-shaped MAX phase). Thereafter, the fired body obtained is pulverized by an end mill to obtain a powdery MAX phase for the next step.

### Step (b)

Etching is performed to remove at least some A atoms from the precursor using an etching liquid. The etching liquid may include, for example, one or more of HF, H₃PO₄, HCl, HI, and H₂SO₄. The etching liquid preferably contains at least one of HF (hydrofluoric acid) and H₃PO₄ (phosphoric acid). For example, it is also possible to perform etching by a so-called MILD method in which HCl and LiF contained in the etching liquid are reacted in a system to generate HF, but preferably, a so-called ACID method in which etching is performed with an etching liquid containing HF (hydrofluoric acid), or a method in which etching is performed with an etching liquid containing phosphoric acid is preferable. According to these methods, as compared with the MILD method, particles (MXene particles) of a flaky layered material having a large flat region with a number-average value of a ferret diameter of preferably not less than 3 μm can be easily obtained, which is preferable. Other conditions for etching are not particularly limited, and known conditions can be adopted. As the etching liquid, a mixed solution of the acid and, for example, pure water as a solvent may be used. As the etching liquid, an etching liquid satisfying at least one selected from the group consisting of an HF concentration of not less than 1.5 M, an H₃PO₄ concentration of not less than 5.5 M, an HCl concentration of not less than 6.0 M, an H₃PO₄ concentration of not less than 5.5 M, an HI concentration of not less than 5.0 M, and an H₂SO₄ concentration of not less than 5.0 M can be used. In the etching of the A atoms, a part of the M atoms may be selectively etched together with the A atoms. Examples of the etched product obtained by the etching include slurry.

### Step (c)

The etched product (first intermediate) obtained by the etching is washed with water. By performing water washing, the acid and the like used in the etching can be sufficiently removed. The amount of water mixed with the etched product and the washing method are not particularly limited. For example, stirring, centrifugation, and the like may be performed by adding water. Examples of the stirring method include stirring using a handshake, an automatic shaker, a share mixer, a pot mill, or the like. The degree of stirring such as stirring speed and stirring time may be adjusted according to the amount, concentration, and the like of the object to be treated. The washing with water may be performed one or more times. Preferably, washing with water is performed multiple times. For example, specifically, steps (i) to (iii) of (i) adding water and stirring (to the etched product or the remaining precipitate obtained in the following (iii)), (ii) centrifuging the stirred product, and (iii) discarding the supernatant after centrifugation and recovering the remaining precipitate are performed within a range of not less than 2 times, for example, not more than 15 times.

### Step (d)

The treated product (second intermediate) obtained by washing with water and the compound for inter-layer insertion of the second intermediate are mixed to perform intercalation.

The compound for inter-layer insertion of the second intermediate may be of any specific type as long as it is a compound that can be inserted between the layers of the second intermediate and can be separated into the respective layers by the delamination treatment in the next step (e). The compound for inter-layer insertion is preferably an alkali metal compound or an alkaline earth metal compound. A Li-containing compound is more preferable. As the Li-containing compound, an ionic compound in which a Li ion and a cation are bonded can be used. Examples of the Li-containing compound include halides including iodide, chloride, and fluoride, phosphates, sulfides including sulfate, nitrates, acetates, and carboxylates.

The content of the compound for inter-layer insertion in the intercalation formulation is preferably 0.001% by mass or more. The content is more preferably 0.01% by mass or more, and still more preferably 0.1% by mass or more. On the other hand, from the viewpoint of dispersibility in a solution, the content of the compound for inter-layer insertion is preferably 10% by mass or less, and more preferably 1% by mass or less.

The specific method of intercalation is not particularly limited, and for example, the compound for inter-layer insertion may be mixed with the moisture medium clay of MXene and stirred, or may be allowed to stand. For example, stirring at room temperature can be mentioned. Examples of the stirring method include a method using a stirring bar such as a stirrer, a method using a stirring blade, a method using a mixer, and a method using a centrifugal device. The stirring time can be set according to the producing scale of the electrode, and may be, for example, set to 12 to 24 hours.

### Step (e)

The delamination including stirring the treated product (third intermediate) obtained by the intercalation in step (d) in a liquid such as, for example, water is carried out. By this delamination, it is possible to realize the single layer/few-layer MXene. The conditions for delamination are not particularly limited, and delamination can be performed by a known method. Examples of the stirring method include stirring using a handshake, an automatic shaker, or the like. The degree of stirring such as stirring speed and stirring time may be adjusted according to the amount, concentration, and the like of the treated product which is an object to be treated. For example, the slurry after the intercalation is centrifuged to discard the supernatant, then pure water is added to the remaining precipitate, and then stirring is performed by, for example, a handshake or an automatic shaker to perform layer separation.

In the producing method of the present embodiment, an ultrasonic treatment is not performed as delamination. As described above, since the ultrasonic treatment is not performed, particle breakage hardly occurs, and as a result, a single layer/few-layer MXene having a large plane parallel to the layer of particles, that is, a large two-dimensional plane, and a large number-average value of a ferret diameter can be obtained as the layered material particles.

### Step (f)

The treated product (fourth intermediate) obtained by delamination is washed with water to obtain particles of a layered material. The amount of water and the washing method are not particularly limited. For example, stirring, centrifugation, and the like may be performed by adding water. Examples of the stirring method include stirring using a handshake, an automatic shaker, a share mixer, a pot mill, or the like. The degree of stirring such as stirring speed and stirring time may be adjusted according to the amount, concentration, and the like of the object to be treated. The washing with water may be performed one or more times. Preferably, washing with water is performed multiple times. For example, specifically, steps (i) to (iii) of (i) adding water and stirring, (ii) centrifuging the stirred product, and (iii) recovering the supernatant after centrifugation are performed within a range of not less than 2 times, for example, not more than 10 times to obtain a MXene-containing supernatant. Thereafter, the MXene-containing supernatant is centrifuged to obtain an MXene-containing clay.

### Step (g)

A conductive film containing particles of the layered material is formed on a substrate. A method for forming the conductive film on the substrate is not limited, and examples thereof include coating and filtration such as suction filtration. As a coating method, for example, in a case where the substrate (for example, a board) is coated with a first slurry (layered material particle-containing slurry) containing the layered material particles, the coating method is not limited. Examples of the coating method include a spray coating method in which spray coating is performed using a nozzle such as a one-fluid nozzle, a two-fluid nozzle, or an air brush, a slit coating method using a table coater, a comma coater, or a bar coater, a screen printing method, a metal mask printing method, a spin coating, and coating methods by immersion, brush, or dropping.

The coating and drying may be repeated a plurality of times as necessary until a conductive film having a desired thickness is obtained. The drying and curing may be performed, for example, at a temperature of 400° C. or lower using a normal pressure oven or a vacuum oven.

As described above, the electrode according to the present embodiment may or may not have a substrate. In the case of an electrode having a substrate, a substrate formed of the above-described material can be used. For example, in a case of using a flexible organic material substrate, plasma treatment may be performed before coating to perform pretreatment for improving the hydrophilicity of the surface. In the case of an electrode having no substrate, the substrate used in this step may be a substrate for forming a conductive film, and the material is not limited. For example, when a conductive film is formed by suction filtration, a polymer film such as a membrane filter can serve as a substrate.

### Step (h)

The π-electron conjugated compound film containing a π-electron conjugated compound having one or more selected from the group consisting of an aromatic ring, a heteroaromatic ring, a carbon-carbon double bond, a carbon-carbon triple bond, and a carbon-phosphorus double bond is formed on the conductive film. As the π-electron conjugated compound, the compound described in the above description of the electrode can be used. For example, a second slurry (π-electron conjugated compound-containing slurry or water dispersion of π-electron conjugated compound) in which the π-electron conjugated compound is dispersed in water is applied onto the conductive film.

In a case where the first slurry (layered material particle-containing slurry) is used for forming the conductive film and the second slurry (r-electron conjugated compound-containing slurry) is used for forming the π-electron conjugated compound film, it is preferable that the conductive film is formed by applying the first slurry containing particles of a layered material in a solid content concentration of not less than 10 mg/mL and not more than 250 mg/mL to the substrate, and the π-electron conjugated compound film is formed by applying the second slurry containing the π-electron conjugated compound in a solid content concentration of not less than 10 mg/mL and not more than 250 mg/mL to the surface of the conductive film.

In addition, in a case where the first slurry (layered material particle-containing slurry) and the second slurry (r-electron conjugated compound-containing slurry) having the above concentrations are used for the production of the electrode, the ratio of the coating amount of the second slurry to the total coating amount of the first slurry and the second slurry is preferably not less than 5% by volume and not more than 80% by volume. When the first slurry and the second slurry satisfy the range of the solid content concentration and the ratio, a stacked film of the conductive film and the π-electron conjugated compound film, which can maintain excellent electrode characteristics, is easily obtained, which is preferable.

The coating method of the π-electron conjugated compound-containing slurry is not limited, and examples thereof include a spray coating method in which spray coating is performed using a nozzle such as a one-fluid nozzle, a two-fluid nozzle, or an air brush, a slit coating method using a table coater, a comma coater, or a bar coater, a screen printing method, a metal mask printing method, a spin coating, and coating methods by immersion, brush, or dropping.

Also in the formation of the r-electron conjugated compound film, the coating and drying may be repeated a plurality of times as necessary until the π-electron conjugated compound film having a desired thickness is obtained. The drying and curing may be performed, for example, at a temperature of 400° C. or lower using a normal pressure oven or a vacuum oven.

Although the electrode and the producing method thereof in the embodiment of the present invention have been described in detail above, various modifications are possible. It should be noted that the electrode according to the present embodiment may be produced by a method different from the producing method in the above-described embodiment, and the method for producing an electrode according to the present embodiment is not limited only to one that provides the electrode according to the above-described present embodiment.

### EXAMPLES

### Example 1

In Example 1, an experiment was conducted to compare temporal changes in impedance between a rGO-MXene laminate (layered composite body) in which rGO (reduced graphene oxide) was stacked as a π-electron conjugated compound film and an rGO non-stacked MXene layer.

### Preparation of Particles of Layered Material

MXene was produced by the following method (ACID method was used as the MAX etching method). Details are as follows.

### Production of MXene Particles Using ACID Method as MAX Etching Method

### (1) Preparation of precursor (MAX)

A TiC powder, a Ti powder, and an Al powder (all available from Kojundo Chemical Laboratory Co., Ltd.) were put in a ball mill containing zirconia balls at a molar ratio of 2:1:1, and mixed for 24 hours. The obtained mixed powder was fired at 1350° C. for 2 hours under an Ar atmosphere. The fired body (block-shaped MAX) thus obtained was pulverized with an end mill to a maximum dimension of not more than 40 μm. In this way, Ti₃AlC₂ particles were obtained as a precursor (powdery MAX)

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.
(Etching Conditions)
Precursor: Ti₃AlC₂ (sieving with a mesh size of 45 μm)
Etching liquid composition: 6 mL of 49% HF,
H₂O 18 mL
HCl (12M) 36 mL
Amount of precursor input: 3.0 g
Etching container: 100 mL IBOY
Etching temperature: 35° C.
Etching time: 24 h
Stirrer rotation speed: 400 rpm
(3) Washing after etching

The slurry was divided into two portions, each of which was inserted into two 50 mL centrifuge tubes, centrifuged under the condition of 3500 G using a centrifuge, and then the supernatant was discarded. An operation of adding 40 mL of pure water to each centrifuge tube, centrifuging again at 3500 G, and separating and removing the supernatant was repeated 11 times. After final centrifugation, the supernatant was discarded to obtain a Ti₃C₂Tₛ-moisture medium clay as a remaining precipitate.

### (4) Li Intercalation

The Ti₃C₂Tₛ-moisture medium clay prepared by the above method was stirred at not lower than 20° C. but not higher than 25° C. for 12 hours using LiCl as a Li-containing compound according to the following conditions of Li intercalation to perform Li intercalation. The detailed conditions of Li intercalation are as follows.

(Conditions of Li Intercalation)
Ti₃C₂Tₛ-moisture medium clay (MXene after washing) solid content 0.75 g
LiCl: 0.75 g
Intercalation container: 100 mL IBOY
Temperature: not lower than 20° C. but not higher than 25° C. (room temperature)
Time: 10 h
Stirrer rotation speed: 800 rpm
(5) Delamination and washing with water

The slurry obtained by Li intercalation was charged into a 50 mL centrifuge tube, centrifuged under the condition of 3500 G using a centrifuge, and then the supernatant was discarded. Next, (i) 40 mL of pure water was added to the remaining precipitate, and the mixture was stirred for 15 minutes with a shaker, then (ii) centrifuged at 3500 G, and (iii) the supernatant was recovered as a single layer/few-layer MXene-containing liquid. The operations (i) to (iii) were repeated four times in total to obtain a single layer/few-layer MXene-containing supernatant.

Further, this supernatant was centrifuged under the conditions of 4300 G and 2 hours using a centrifuge, and then the supernatant was discarded to obtain a single layer/few-layer MXene-containing clay as a remaining precipitate.

### Formation of Electrode Sample

Pure water was added to the single layer/few-layer MXene-containing clay to obtain MXene slurry (MXene aqueous dispersion) in which the solid content concentration of the particles of the layered material was about 10 mg/mL. The MXene slurry was spray-coated once on a slide glass and dried to prepare a MXene thin film having a thickness of 0.1 μm.

Subsequently, an aqueous dispersion of reduced graphene oxide (rGO) having a solid content concentration of about 15 mg/mL was spray-coated twice on the MXene thin film, then dried to form an rGO film having a thickness of 0.2 μm as a π-electron conjugated compound film, and prepare an rGO-MXene stacked film as an electrode sample.

(Measurement of Initial Impedance and Change Rate)

The electrode formed of the rGO-MXene stacked film was immersed in phosphate buffered saline (PBS) heated to 73° C. in a beaker for 24 days, and the impedance was periodically measured. The impedance was measured at the time of immersion (Day 0) and the immersion elapsed days shown in Tables 2 and 3. The impedance measurements were performed using a Gamry Reference 600 Potentiostat/Galvanostat/ZRA in a standard three-electrode system using a saturated Ag/AgCl reference electrode (Sigma-Aldrich Co. LLC; 3 M potassium chloride) and a carbon counter electrode. The electrochemical impedance spectroscopy (EIS) was in a range of 0.1 to 105 Hz and the driving voltage was 10 mV (Peak-to-peak amplitude, sine wave). The impedance was determined at each of 10 Hz and 1 kHz.

In order to obtain the reference value, instead of the rGO-MXene stacked film, a sample in which only the MXene layer, that is, the rGO non-stacked MXene layer was formed as described above was also prepared, and the same measurement was performed. Then, the initial impedance (Day 0) of the rGO non-stacked MXene layer was set to 1.00, the ratio of each measured impedance value to the initial impedance of the rGO non-stacked MXene layer was obtained as an impedance change rate, and the temporal change of the impedance was evaluated. The initial impedance values of the rGO non-stacked MXene layer and the rGO-MXene stacked film are shown in Table 1. In Table 1, in the measured value of the rGO-MXene stacked film, the impedance change rate with respect to the initial impedance of the rGO non-stacked MXene layer is also shown in parentheses. In addition, as the temporal change of the impedance, the results of measuring the impedance on the elapsed days shown in Tables 2 and 3 are shown in Table 2 in the case of 10 Hz and in Table 3 in the case of 1 kHz. In Tables 2 and 3, the numerical values in parentheses are the change rate of each impedance with respect to the initial impedance of the rGO-MXene stacked film.

TABLE 1

| | Initial impedance (Ω · cm²) | |
|---|---|---|
| | @10 Hz | @1 kHz |
| rGO non-stacked | 17.82 | 15.25 |
| MXene layer | | |
| rGO-MXene laminate | 40.87 | 34.6 |
| | (Change rate: 2.29) | (Change rate: 2.27) |

TABLE 2

| | Impedance change rate | |
|---|---|---|
| | @10 Hz | |
| Elapsed | rGO non-stacked | rGO-MXene |
| date | MXene layer | laminate |
| 0 | 1.00 | 2.29 (1.00) |
| 3 | 6.01 | 3.95 (1.72) |
| 6 | 7.23 | 8.75 (3.82) |
| 9 | 14.03 | 8.27 (3.61) |
| 12 | 7.41 | 8.06 (3.52) |
| 15 | 9.98 | 9.38 (4.10) |
| 18 | 15.21 | 9.36 (4.09) |
| 22 | 19.08 | 10.98 (4.79) |
| 24 | 12.68 | 8.37 (3.66) |

TABLE 3

| | Impedance change rate | |
|---|---|---|
| | @1 KHz | |
| Elapsed | rGO non-stacked | rGO-MXene |
| date | MXene layer | laminate |
| 0 | 1.00 | 2.27 (1.00) |
| 3 | 2.14 | 3.63 (1.60) |
| 6 | 2.67 | 4.77 (2.10) |
| 9 | 4.28 | 4.49 (1.98) |
| 12 | 3.46 | 3.76 (1.66) |
| 15 | 2.93 | 4.31 (1.90) |
| 18 | 3.58 | 3.21 (1.41) |
| 22 | 4.12 | 5.27 (2.32) |
| 24 | 3.99 | 3.78 (1.67) |

As shown in Table 1, the initial impedance of the rGO-MXene stacked film was about twice as large as that of the rGO non-stacked MXene layer, but the impedance of the rGO non-stacked MXene layer was more rapidly increased than that of the rGO-MXene stacked film with the lapse of time and date. As a result, in the case of 10 Hz, the impedance on the 24 elapsed days was more than 12 times the initial impedance in the rGO non-stacked MXene layer; whereas it was about 8 times in the case of the rGO-MXene stacked film, and the increase was suppressed as compared with the rGO non-stacked MXene layer. In addition, in the case of 1 kHz, the rGO non-stacked MXene layer increased about four times the initial impedance; whereas the rGO-MXene stacked film increased less than four times the initial impedance. In this case, the increase was suppressed as compared with the rGO non-stacked MXene layer. Further, when the initial impedance of each of the rGO non-stacked MXene layer and the rGO-MXene stacked film was set to 1.00, in the case of 10 Hz, the impedance change rate was about 12 times after 24 days in the rGO non-stacked MXene layer; whereas the change rate was about 4 times in the rGO-MXene stacked film and was suppressed to about ⅓. Also in the case of 1 kHz, in the rGO-MXene stacked film, the impedance change rate was suppressed to half or less of that of the rGO non-stacked MXene layer. From these results, it is found that in the rGO-MXene stacked film, the temporal change of the impedance is sufficiently suppressed.

Regarding the reason why the rGO-MXene stacked film was able to suppress the temporal change in impedance as compared with the rGO non-stacked MXene layer, it is considered that since rGO has a large amount of n electrons, the metal atoms of MXene and the n electrons of the rGO interact with each other to cause a phenomenon called n bond that enhances conductivity, and it is considered that oxidation of MXene coated with rGO is prevented by oxidation resistance of rGO, and an effect of maintaining the conductivity is exhibited by securing a conductive path by r bond with the MXene.

### Example 2

In Example 2, the influence of the particle size of the layered material on the temporal change of the impedance was examined.

A MXene moisture medium clay was prepared in the same manner as in Example 1. Separately from this, a MXene moisture medium clay was prepared by the following method (production of MXene particles using MILD method as MAX etching method).

### Production of MXene Particles Using MILD Method as MAX Etching Method

### (1) Preparation of Precursor (MAX)

A TiC powder, a Ti powder, and an Al powder (all available from Kojundo Chemical Laboratory Co., Ltd.) were put in a ball mill containing zirconia balls at a molar ratio of 2:1:1, and mixed for 24 hours. The obtained mixed powder was fired at 1350° C. for 2 hours under an Ar atmosphere. The fired body (block-shaped MAX) thus obtained was pulverized with an end mill to a maximum dimension of not more than 40 μm. In this way, Ti₃AlC₂ particles were obtained as a precursor (powdery MAX)

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

(Etching Conditions)
Precursor: Ti₃AlC₂ (sieving with a mesh size of 45 μm)
Etching liquid composition: LiF 3.0 g
H₂O 7.5 mL
HCl (12M) 22.5 mL
Amount of precursor input: 3.0 g
Etching container: 100 mL IBOY
Etching temperature: 35° C.
Etching time: 72 h
Stirrer rotation speed: 400 rpm

### (3) Washing after Etching

The slurry was divided into two portions, each of which was inserted into two 50 mL centrifuge tubes, centrifuged under the condition of 3500 G using a centrifuge, and then the supernatant was discarded. An operation of adding 40 mL of pure water to each centrifuge tube, centrifuging again at 3500 G, and separating and removing the supernatant was repeated 11 times. After final centrifugation, the supernatant was discarded to obtain a Ti₃C₂Tₛ-moisture medium clay as a remaining precipitate.

### (4) Delamination

Next, (i) 40 mL of pure water was added to a Ti₃C₂Tₛ-moisture medium clay, and the mixture was stirred for 15 minutes with a shaker, then (ii) centrifuged at 3500 G, and (iii) the supernatant was recovered as a single layer/few-layer MXene-containing liquid. The operations (i) to (iii) were repeated four times in total to obtain a single layer/few-layer MXene-containing supernatant. Further, this supernatant was centrifuged under the conditions of 4300 G and 2 hours using a centrifuge, and then the supernatant was discarded to obtain a single layer/few-layer MXene-containing clay as a remaining precipitate.

Pure water was added to the single layer/few-layer MXene-containing clay of 10 mM to prepare a MXene moisture medium clay (MILD method) having a solid content concentration of not more than 10 mg/mL.

The MXene slurry obtained by diluting the two kinds of MXene moisture medium clays with pure water was spray-coated on a slide glass and dried to prepare a MXene thin film.

(Measurement of Initial Impedance of Each MXene Thin Film)

The electrode formed of the MXene thin film produced by each method was allowed to stand in the air at room temperature until 12 weeks elapsed, and the impedance was measured at weeks 2, 4, 6, 7, 10, and 12. The impedance was measured in the elapsed weeks shown in Table 5. The impedance measurement conditions were the same as in Example 1.

(Measurement of Number-Average Value of a Ferret Diameter of Particles of Layered Material Constituting Each MXene Thin Film)

In both the layered material particles obtained by etching MAX by the ACID method and the layered material particles obtained by the MILD method, the number-average value of a ferret diameter was measured by removing the rGO layer from the rGO-MXene stacked film. Specifically, the rGO layer was broken by polishing from the rGO-MXene stacked film, and the remaining film was subjected to ultrasonic cleaning in pure water to disperse the MXene flakes in pure water. An optional amount of pure water in which the MXene flakes were dispersed was dropped on a Si substrate. Then, the Si substrate supporting MXene flakes was obtained by allowing the Si substrate to stand at room temperature for 8 hours and remove moisture. Then, the MXene flakes supported on the Si substrate were photographed at a magnification of 2000 times using a scanning electron microscope (product name: S-4800) manufactured by Hitachi High-Technologies Corporation, and the obtained photograph was visually observed and confirmed. For the number-average value of a ferret diameter, 50 or more particles were optionally extracted from the photograph, the Feret (Feret) constant direction tangent diameter of each particle was determined, and the average value of the number was determined as the number-average value of a ferret diameter.

As a result of calculating the number-average value of a ferret diameter, when the etching of MAX was performed by the MILD method, the number-average value of a ferret diameter was less than 3.00 μm, and when the etching of MAX was performed by the ACID method, the number-average value of a ferret diameter was 3.00 μm or more.

Even in a case of a method in which the etching of MAX is performed by the following phosphoric acid etching instead of the ACID method described in Example 1, it is considered that the MXene particles having a number-average value of a ferret diameter of 3.00 μm or more are obtained and an effect of suppressing an increase in impedance is obtained.

### Method Including Phosphoric Acid Etching of MAX

### Preparation of Sample

The MXene particles were prepared by sequentially performing the following five steps described in detail below: (1) preparation of the precursor (MAX), (2) etching of the precursor, (3) washing, (4) intercalation, and (5) delamination.

### (1) Preparation of Precursor (MAX)

A TiC powder, a Ti powder, and an Al powder (all available from Kojundo Chemical Laboratory Co., Ltd.) were put in a ball mill containing zirconia balls at a molar ratio of 2:1:1, and mixed for 24 hours. The obtained mixed powder was fired at 1350° C. for 2 hours under an Ar atmosphere. The fired body (block) thus obtained was pulverized with an end mill to a maximum dimension of not more than 40 μm. In this way, Ti₃AlC₂ particles were obtained as a precursor (MAX).

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

(Etching Conditions)
Precursor: Ti₃AlC₂ (sieving with a mesh size of 45 μm)
Etching liquid composition: Aqueous solution having HF concentration of not less than 1.5 M and H₃PO₄ concentration of not less than 5.5 M
Amount of precursor input: 3.0 g
Etching container: 100 mL IBOY
Etching temperature: 35° C.
Etching time: 24 h
Stirrer rotation speed: 400 rpm

### (3) Washing

The slurry was divided into two portions, each of which was inserted into two 50 mL centrifuge tubes, centrifuged under the condition of 3500 G using a centrifuge, and then the supernatant was discarded. (i) In each centrifuge tube, 40 mL of pure water was added to the clay precipitated by centrifugation and mixed to form a slurry,
(ii) The step of centrifuging again at 3500 G to separate and remove the supernatant was repeated 11 times. After final centrifugation, the supernatant was discarded to obtain a Ti₃C₂Tₛ-moisture medium clay as a remaining precipitate.

### (4) Intercalation

Li₃PO₄, H₃PO₄, and pure water were added to the Ti₃C₂Tₛ-moisture medium clay prepared by the above method, and the mixture was stirred at not lower than 20° C. but not higher than 25° C. for 15 hours to perform Li intercalation. The detailed conditions of Li intercalation are as follows.

(Conditions of Li Intercalation)
Ti₃C₂Tₛ-moisture medium clay (MXene after washing) solid content 0.75 g
Li₃PO₄: 0.68 g
85% by mass of H₃PO₄: 3.1 mL
H₂O: 31.9 mL
Intercalation container: 100 mL IBOY
Temperature: not lower than 20° C. but not higher than 25° C. (room temperature)
Time: 15 h
Stirrer rotation speed: 800 rpm

### (5) Delamination

The slurry after the Li intercalation was charged into a 50 mL centrifuge tube, centrifuged under the condition of 3500 G using a centrifuge, and then the supernatant was discarded. Subsequently, an operation of adding 40 mL of pure water to the remaining precipitate from which the supernatant was removed, stirring the mixture with a shaker for 15 minutes, then centrifuging the mixture at 3500 G, and recovering the supernatant as a single layer/few-layer MXene-containing liquid was repeated 4 times to obtain a single layer/few-layer MXene-containing sample.

The results of measuring the initial impedance at 10 Hz and 1 kHz for each of the number-average value of a ferret diameters are shown in Table 4. For the change rate in parentheses in Table 4, the reduction ratio of the initial impedance due to an increase in the ferret diameter from less than 3.00 μm to not less than 3.00 μm is indicated by a negative value. Further, the temporal change in impedance is shown in Table 5 for the case of 10 Hz and Table 6 for the case of 1 kHz for each of the number-average value of a ferret diameter of less than 3.00 μm and not less than 3.00 μm.

TABLE 4

| Number-average value | Initial impedance (Ω · cm²) | |
|---|---|---|
| of ferret diameter | @10 Hz | @1 kHz |
| Less than 3.00 μm | 11.69 | 3.36 |
| Not less than 3.00 μm | 10.37 | 3.21 |
| | (Change rate: −11.25%)] | (Change rate: −4.41%) |

TABLE 5

| | Impedance change rate | |
|---|---|---|
| | @10 Hz | |
| | Number-average value | Number-average value |
| Elapsed | of ferret diameter is | of ferret diameter is not |
| date | less than 3.00 μm | less than 3.00 μm |
| 0 | 1.00 | 0.89 |
| 2 | 2.20 | 1.59 |
| 4 | 4.50 | 2.61 |
| 6 | 5.90 | 3.07 |
| 7 | 6.76 | 3.94 |
| 10 | 6.73 | 4.34 |
| 12 | 7.23 | 4.25 |

TABLE 6

| | Impedance change | |
|---|---|---|
| | rate @1 kHz | |
| | Number-average value | Number-average value |
| Elapsed | of ferret diameter is | of ferret diameter is |
| date | less than 3.00 μm | not less than 3.00 μm |
| 0 | 1.00 | 0.96 |
| 2 | 1.60 | 1.51 |
| 4 | 2.07 | 1.91 |
| 6 | 2.49 | 1.97 |
| 7 | 2.55 | 2.15 |
| 10 | 2.71 | 2.20 |
| 12 | 2.92 | 2.30 |

It is found that the initial impedance can be reduced by about −10% to −5% by applying MXene having a number-average value of a ferret diameter of 3.00 μm or more as shown in Table 4. This is considered to be due to the improvement of the in-plane conductivity because one MXene sheet is large when produced by the ACID method. As shown in Table 5, the impedance after 12 weeks (at 10 Hz) also moderately increased when the number-average value of a ferret diameter of the particles was 3.00 μm or more. Specifically, from the comparison of the impedance change rate on 12 weeks elapsed between the case where the number-average value of a ferret diameter is 3.00 μm or more and the case where the number-average value of a ferret diameter is less than 3.00 μm in Table 5, the impedance change rate was able to be suppressed to about half. This effect is considered to be due to a reduction in the number of oxidation sites (the number of edges) per unit area of the particles as the number-average value of a ferret diameter is 3.00 μm or more.

The MXene shown in Tables 1 and 2 was obtained by performing etching by the ACID method, and the number-average value of a ferret diameter of the particles is 3 μm or more. From Tables 1 and 2, it is found that the impedance change rate can be sufficiently suppressed by using MXene particles having a large ferret diameter and stacking the rGO layers.

The following can be said from these results. In the MXene particles (particles of a layered material), n bond can be formed only in the X-Y plane of the MXene particles, and therefore it is considered that more n bond is formed in particles having a large ferret diameter. It can be said that by forming the conductive film using the particles (MXene particles) of the layered material having a large ferret diameter, preferably a number-average value of a ferret diameter of 3 μm or more, in which more n bonds are easily formed, in addition to the effect by stacking rGO described in Example 1, the in-plane orientation as the MXene film was improved, the formation of the n bond with the rGO to be stacked was facilitated, and the effect of suppressing the increase in impedance became more remarkable.

### Example 3

In Example 3, the influence of the difference in the existence form of rGO on the initial impedance was examined.

### Production of rGO-MXene Stacked Film

An rGO-MXene stacked film was obtained in the same manner as in the rGO-MXene stacked film of Example 1 except that an rGO aqueous dispersion having a solid content concentration of rGO of about 10 mg/mL was used, and the spray coating of each of the MXene slurry (MXene aqueous dispersion) and the rGO aqueous dispersion was performed three times.

### Production of rGO-MXene Mixed Film

A mixed film of rGO and MXene was produced as follows. That is, 10 mL of each of the MXene moisture medium clay having a solid content concentration of MXene of about 10 mg/mL and the rGO aqueous dispersion having a solid content concentration of rGO of about 10 mg/mL were weighed and mixed to produce a mixed solution. This was spray-coated six times to produce an rGO-MXene mixed film. Unlike the rGO-MXene stacked film, the rGO-MXene mixed film is a film in which rGO and MXene are randomly mixed.

(Measurement of Initial Impedance)

The electrode formed of each structure of the rGO-MXene stacked film and the rGO-MXene mixed film was immersed in PBS at room temperature to measure the initial impedance. Other impedance measurement conditions were the same as in Example 1. The initial impedance of each structure in PBS is shown in Table 7.

TABLE 7

| | Initial impedance (Ω · cm²) | |
|---|---|---|
| | @10 Hz | @ 1 kHz |
| rGO-MXene | 11.68 | 3.3 |
| stacked film | | |
| rGO-MXene | 19.59 | 3.49 |
| mixed film | (1.68 times rGO-MXene | (1.06 times rGO-MXene |
| | stacked film) | stacked film) |

As shown in Table 7, the initial impedance of the rGO-MXene mixed film increased up to 1.7 times the initial impedance of the rGO-MXene stacked film. This is considered to be because in the case of the rGO-MXene stacked film (covered type), ionic conduction between layers specific to MXene is not inhibited, the conductivity of MXene is maintained, and deterioration over time due to oxidation can be suppressed; whereas in the case of the rGO-MXene mixed film, the ionic conduction between layers specific to MXene is greatly inhibited, and the initial impedance increases.

### Example 4

In Example 4, the influence of the ratio of the rGO layer to the MXene layer on the initial impedance was examined.

### Production of rGO-MXene Stacked Films Having Different rGO Volume Ratios

First, in the same manner as in Example 1, the rGO aqueous dispersion was spray-coated twice to obtain a rGO-MXene stacked film. In addition, a rGO-MXene stacked film produced in the same manner as in Example 1 except that the number of times of spray coating of the rGO aqueous dispersion was changed to six times was also obtained.

(Measurement of Initial Impedance)

Electrodes formed of rGO-MXene stacked films with two and six spray coating times of the rGO aqueous dispersion, respectively, were immersed in PBS at room temperature in a beaker, and initial impedances at 10 Hz and 1 kHz, respectively, were measured. Other impedance measurement conditions were the same as those in Example 1. The measurement results using each rGO-MXene stacked film are shown in Table 8.

TABLE 8

| | Initial impedance (Ω · cm²) | |
|---|---|---|
| | @10 Hz | @1 kHz |
| rGO-67% by mass-MXene | 61 | 3.96 |
| stacked film | | |
| (Coating twice with rGO | | |
| moisture medium) | | |
| rGO-85% by mass-MXene | 165.2 | 51.2 |
| stacked film | (2.71 times the | (12.93 times the |
| (Coating six times with | above value) | above value) |
| rGO moisture medium) | | |

As shown in Table 8, the initial impedance of the rGO-MXene stacked film in which the proportion of rGO was 67% by mass was suppressed to be sufficiently lower than the initial impedance of the rGO-MXene stacked film in which the proportion of rGO was 85% by mass. From this result, it is considered that when the ratio of rGO in the rGO-MXene stacked film is preferably 80% by mass or less, excellent conductivity due to MXene is exhibited, an increase in initial impedance is suppressed, and deterioration over time due to oxidation of MXene can be suppressed.

The electrode according to the present embodiment can be used for any appropriate application, and can be preferably used as, for example, a biosignal sensing electrode or the like.

### REFERENCE SIGNS LIST

1a, 1b Layer body (MₘXₙ layer)
3a, 5a, 3b, 5b Modifier or terminal T
7a, 7b MXene layer
10a, 10b MXene particles (particles of layered material)
21 Substrate
22 Conductive film (film containing MXene particles)
23 π-electron conjugated compound film
24 Bond between n electron of MXene particle and n electron of π-electron conjugated compound film
25 Biological tissue
26 Electrode

1. An electrode comprising:
a π electron conjugated compound film which comprises a n electron conjugated compound having at least one selected from the group consisting of an aromatic ring, a heteroaromatic ring, a carbon-carbon double bond, a carbon-carbon triple bond, and a carbon-phosphorus double bond; and
a conductive film bonded to the n electron conjugated compound film, wherein the conductive film comprises particles of a layered material comprising one or plural layers, the one or plural layers comprising a layer body represented by:
MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is at least one selected from the group consisting of a hydroxyl group, a fluorine atom, a chlorine atom, an oxygen atom, and a hydrogen atom, and
wherein a ratio of an amount of the n electron conjugated compound film to a total amount of the conductive film and the n electron conjugated compound film is not less than 5% by mass and not more than 80% by mass.

2. The electrode according to claim 1, wherein the r electron conjugated compound is at least one compound selected from the group consisting of a graphene, a graphene oxide, a reduced graphene oxide, a carbon nanotube, a polyacetylene, a polyparaphenylene, a polyparaphenylene vinylene, a polypyrrole, a polythiophene, a polyethylenedioxythiophene, a polyaniline, and a polythienylene vinylene.

3. The electrode according to claim 1, wherein a thickness of the π-electron conjugated compound film is not less than 0.03 μm and not more than 30 μm.

4. The electrode according to claim 1, wherein an area of at least 80% of a surface of the conductive film on a side thereof facing the n electron conjugated compound film is covered with the n electron conjugated compound film.

5. The electrode according to claim 4, wherein a number-average value of a ferret diameter of the particles of the layered material is not less than 3 μm.

6. The electrode according to claim 1, wherein a number-average value of a ferret diameter of the particles of the layered material is not less than 3 μm.

7. The electrode according to claim 1, wherein the electrode is constructed as a biosignal sensing electrode.

8. The electrode according to claim 2, wherein the electrode is constructed as a biosignal sensing electrod.

9. An electrode comprising:
a π electron conjugated compound film which comprises a n electron conjugated compound having at least one selected from the group consisting of an aromatic ring, a heteroaromatic ring, a carbon-carbon double bond, a carbon-carbon triple bond, and a carbon-phosphorus double bond; and
a conductive film bonded to the n electron conjugated compound film, wherein the conductive film comprises particles of a layered material comprising one or plural layers, the one or plural layers comprising a layer body represented by:
MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is at least one selected from the group consisting of a hydroxyl group, a fluorine atom, a chlorine atom, an oxygen atom, and a hydrogen atom, and
wherein a number-average value of a ferret diameter of the particles of the layered material is not less than 3 μm.

10. The electrode according to claim 9, wherein the r electron conjugated compound is at least one compound selected from the group consisting of a graphene, a graphene oxide, a reduced graphene oxide, a carbon nanotube, a polyacetylene, a polyparaphenylene, a polyparaphenylene vinylene, a polypyrrole, a polythiophene, a polyethylenedioxythiophene, a polyaniline, and a polythienylene vinylene.

11. The electrode according to claim 9, wherein an area of at least 80% of a surface of the conductive film on a side thereof facing the n electron conjugated compound film is covered with the n electron conjugated compound film.

12. The electrode according to claim 9, wherein the electrode is constructed as a biosignal sensing electrode.

13. An electrode comprising:
a π electron conjugated compound film which comprises a n electron conjugated compound having at least one selected from the group consisting of an aromatic ring, a heteroaromatic ring, a carbon-carbon double bond, a carbon-carbon triple bond, and a carbon-phosphorus double bond; and
a conductive film bonded to the r electron conjugated compound film, wherein the conductive film comprises particles of a layered material comprising one or plural layers, the one or plural layers comprising a layer body represented by:
MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is at least one selected from the group consisting of a hydroxyl group, a fluorine atom, a chlorine atom, an oxygen atom, and a hydrogen atom, and
wherein an area of at least 80% of a surface of the conductive film on a side thereof facing the n electron conjugated compound film is covered with the n electron conjugated compound film.

14. The electrode according to claim 13, wherein the r electron conjugated compound is at least one compound selected from the group consisting of a graphene, a graphene oxide, a reduced graphene oxide, a carbon nanotube, a polyacetylene, a polyparaphenylene, a polyparaphenylene vinylene, a polypyrrole, a polythiophene, a polyethylenedioxythiophene, a polyaniline, and a polythienylene vinylene.

15. The electrode according to claim 13, wherein the electrode is constructed as a biosignal sensing electrode.

16. The electrode according to claim 14, wherein the electrode is constructed as a biosignal sensing electrode.

17. A method for producing an electrode, the method comprising:
(a) preparing a precursor of a conductive film, the precursor represented by:
MₘAXₙ
wherein M is at least one metal of Group 3, 4, 5, 6, or 7, X is a carbon atom, a nitrogen atom, or a combination thereof, A is at least one element of Group 12, 13, 14, 15, or 16, n is not less than 1 and not more than 4, and m is more than n but not more than 5;
(b) removing at least a part of the A atoms from the precursor using an etching liquid to obtain a first intermediate;
(c) washing the first intermediate with water to obtain a second intermediate;
(d) performing an intercalation by mixing the second intermediate with a compound for inter-layer insertion of the second intermediate to obtain a third intermediate;
(e) stirring the third intermediate in a liquid to obtain a fourth intermediate;
(f) washing the fourth intermediate with water to obtain particles of a layered material;
(g) coating a substrate with a first slurry comprising the particles of the layered material at a solid content concentration of not less than 10 mg/mL and not more than 250 mg/mL to form a conductive film comprising the particles of the layered material on the substrate; and
(h) coating a surface of the conductive film with a second slurry comprising a π electron conjugated compound at a solid content concentration of not less than 10 mg/mL and not more than 250 mg/mL so as to form a π electron conjugated compound film on the conductive film, the n electron conjugated compound having at least one selected from the group consisting of an aromatic ring, a heteroaromatic ring, a carbon-carbon double bond, a carbon-carbon triple bond, and a carbon-phosphorus double bond.

18. The method for producing the electrode according to claim 17, wherein the etching liquid comprises at least one of hydrofluoric acid or phosphoric acid.

19. The method for producing the electrode according to claim 17, wherein the n electron conjugated compound is at least one compound selected from the group consisting of a graphene, a graphene oxide, a reduced graphene oxide, a carbon nanotube, a polyacetylene, a polyparaphenylene, a polyparaphenylene vinylene, a polypyrrole, a polythiophene, a polyethylenedioxythiophene, a polyaniline, and a polythienylene vinylene.

20. The method for producing the electrode according to claim 17, wherein a ratio of a coating amount of the second slurry to a total coating amount of the first slurry and the second slurry is not less than 5% by volume and not more than 80% by volume.
